(19) United States
(12) Patent Application Publication
(10) Pub. No.: US 2021/0025860 A1
(43) Pub. Date: Jan. 28, 2021

(54) SENSOR LAYER SYSTEM PRECURSOR, SENSOR LAYER SYSTEM WHICH CAN BE PRODUCED THEREFROM, HYDROGEN SENSOR ELEMENT WHICH USES SAID SENSOR LAYER SYSTEM, AND CORRESPONDING PRODUCTION METHOD

(71) Applicant: Trafag AG, Schweiz (CH)

(72) Inventor: Wendelin J. STARK, Langenthal (DE)

(21) Appl. No.: 17/042,882

(22) PCT Filed: Mar. 28, 2019
(86) PCT No.: PCT/EP2019/057826

§ 371 Date: Sep. 28, 2020

(30) Foreign Application Priority Data
Mar. 28, 2018 (DE) 10 2018 107 516.2

(51) Int. Cl. G01N 33/00

(52) U.S. Cl. CPC G01N 33/005

(57) ABSTRACT

A sensor layer system precursor (48) configured for forming a sensor layer system (26), the sensor layer system (26) being configured for absorbing hydrogen, includes a sensing layer precursor (42) made from a sensing layer precursor material that consists of: 20% by weight to 90% by weight palladium or palladium alloy, the palladium alloy consisting of palladium and at least one palladium alloy partner chosen from group VIIIB, wherein the amount-of-substance fraction of palladium is at least 85% and the sum of the amount-of-substance fractions of all palladium alloy partners contained in the palladium alloy is at most 15% with respect to the whole amount of substance of the palladium alloy, respectively; 10% by weight to 80% by weight sacrificial metal, the sacrificial metal being at least as electropositive as palladium and each palladium alloy partner and/or the sacrificial metal being selectively transformable by a chemical process into a soluble and/or ionic form; remainder unavoidable impurities; and optionally up to and including 30% by weight pore filler precursor metal that is transformable into a pore filler by means of a pore filler reaction component.

The invention relates to a sensor layer system precursor, a sensor layer system that is manufacturable therefrom as well as a hydrogen sensor element using the same and corresponding manufacturing methods.

Hydrogen concentration in a gas is of high technical interest on the one hand in monitoring safety of a plant or operation, on the other hand in controlling plants that use hydrogen (e.g. hydrogen fuel cells) or generate hydrogen (e.g. manufacturing of hydrogen for energy storage). The hydrogen concentration in liquids is of high technical interest in monitoring operation of chemical plants that use hydrogen for hydrogenation, in fuel cells as well as in process monitoring of organic lubrication and heat transferring media where hydrogen concentration indicates decomposition of the medium, and thus a change of medium or replacement may be initiated before critical mechanical failure may occur.

Hydrogen concentration in gases is usually measured in fraction per volume in the gas. For high fractions per volume the concentration is measured in percent per volume (% V/V), for lesser hydrogen concentration the concentration is measure in parts per million (ppm). The hydrogen concentration of air is of particular interest, since higher hydrogen concentrations (4% to 75% at standard pressure and room temperature) may form flammable and often explosive (from 17%) mixtures, which caused numerous and often tragic accidents in the past. In partially or completely closed-off areas typically the hydrogen concentration is monitored such that the concentration of hydrogen is below ten percent of the lower explosion limit (i.e. 0,4% corresponding to 4100 ppm). For higher concentrations the US Environmental Protection Agency (US EPA) recommends evacuation of personnel from such areas.

A frequent reason for the use of hydrogen sensors is the rapid spread of hydrogen in air and the often large amounts of hydrogen that may be present in corresponding processes or plant parts. It is thus of high interest to initialize rapid correction measures (e.g. closing of a valve of the hydrogen supply) or emergency measures (e.g. ventilating the area or switching of the plant) if the hydrogen concentration is rising.

During measurement of hydrogen in liquids the measurement speed is also of high importance since decomposition of lubrication or heat transfer media typically indicates impending failure of a part or bearing, where serious damages may often be only avoided by a rapid switch off of plant parts or replacement of heat transfer media.

The measurement may be performed by multiple basically different measurement principles.

In the presence of oxygen (or other oxidizing gases) detection may be performed by means of a frequently metallic sensor element that is heated, and thus catalytically oxidizes the hydrogen in the gas mixture to be measured. The locally generated heat during this can be measured (typically as a temperature difference) and be used a basis for determining the hydrogen concentration. The sensor elements are often chosen from group VIIIB (groups 8, 9 and 10, respectively), preferably nickel palladium and platinum. These so called hot wire sensors have considerable difficulty in distinguishing hydrogen from other reducing gases. The latter may also be oxidized on the sensor and generate heat. Due to this inaccurate measurement data may be obtained, in particular in the presence of hydrocarbons, carbon monoxide, formaldehyde, methanol, ethanol or other organic compounds which are widely used. Resulting false alarms are expensive since they may cause interruption of processes or standstill. A second technical problem is the dependence of the operation of the sensor on the availability of an oxidizer. As a result these hot wire sensors may operate worse or in extreme cases not at all, if the gas to be measured includes few or no oxygen. Liquids that are monitored for their hydrogen concentration often lack oxygen. The same is true for most gas mixtures which for reasons of corrosion or security considerations avoid oxygen or do not include oxygen at all.

U.S. Pat. No. 6,029,500 A discloses determining hydrogen in absence of oxidizer (typically oxygen). This idea is based on an oscillating member that is coated with a palladium containing metal film. The use of 250 angstrom (25 nanometer) thick palladium layers allows sensors that are able to sense hydrogen in a range less than one percent to pure hydrogen. The sensor response is small, however. The use of a 1000 angstrom (100 nanometer, 100 nm) palladium layer increases the sensor response to a frequency change of 400 hertz during change from no (so 0% hydrogen in gas) to pure (so 100%) hydrogen. This is enough for reliable detection (yes/no response with respect to presence of hydrogen in the percent range) but of limited accuracy (e.g. distinguishing 2000 ppm and 2500 ppm) and does not allow for sensing of low hydrogen concentrations (1 to 100 ppm). The use of 250 angstrom (25 nm) palladium film sensor exhibit a temperature drift of 16 hertz per degree centigrade and a hydrogen sensitivity of 30 hertz per percent change of hydrogen concentration. With this the accuracy of these sensors is insufficient for most applications, since a temperature change of 2 degrees centigrade generates a larger sensor response (2×16 hertz=32 hertz) than the change of the hydrogen content by 1 percentage point (30 hertz). Monitoring of hydrogen storages, fuel cells and batteries are subject to much larger temperature variations, in particular in transportation, so that such sensors for themselves are of limited practical interest.

Thus, the sensors of the above type often require additional reference systems which have identical configuration but instead of the active (palladium containing) layer include a non-active reference layer (e.g. gold). Double oscillator members are needed however which has increased effort regarding space, costs, calibration and maintenance. While thicker palladium layers can increase the sensor response in principle, the response time may also be increased unacceptably: the response time, in particular with decreasing hydrogen concentration, of 1000 angstrom (100 nm) palladium layer sensors is in the range of minutes which is considerably too slow in many technical applications. Reference is made to Einstein's equation:

d=t²/D

wherein d is the diffusion distance (in meters), t the time (in seconds), during which the diffusion takes place and D the diffusion constant (in square meters per second).

Consequently, thicker palladium layers (above 100 nm) react very slowly (response times of multiple minutes) and are of limited practical interest.

U.S. Pat. No. 7,647,813 B2 discloses another disadvantage of such palladium films on quartz oscillators namely the worse stability of such systems, in particular due to poisoning of the palladium surface, e.g. by sulfur or sulfuric compounds, and due to delamination of the palladium layers as a result of the large change of volume (above 1% change of volume by absorption of hydrogen in palladium) during hydrogen absorption and discharge, in particular for higher concentrations and frequent changes of concentration. These may cause enormous mechanical tension between the substrate of the palladium metal film (no change of volume due to changing hydrogen concentration, typically quartz) and a palladium film (large change of volume). Changing hydrogen concentration may generate cracks. This can cause insufficient bonding of the palladium containing film to the part, which may be expressed by frequency change and shift or signal drift; in the extreme delamination of the palladium containing layer or complete loss of function of the sensor may occur. Since long term stability is paramount (high cost for maintenance of or replacing a sensor) a pair of oscillating members (OSC1 and OSC2) is used, wherein one oscillating member is exposed to the hydrogen containing gas and the reference element is not. In order to obtain reasonable results, the reference gas should be maintained at the same pressure, temperature and composition (except hydrogen) as the gas to be measured.

The large volume change deemed unfavorable in U.S. Pat. No. 7,647,813 B2 can also be used for detecting as disclosed in WO 2007/0 019 244 A1. Therein, palladium containing metal nanoparticles are deposited on a film and the change of electric resistance of the layer is measured after significant calibration effort. With high hydrogen concentration the resistance decreases due to the particles being enlarged by the absorbed hydrogen. The response time (time to achieve 90% of a stable final value) of these sensors is between 3 minutes and an hour at 4000 ppm hydrogen. The sensor response is in addition heavily dependent on temperature, wherein a temperature change of 10 degrees centigrade generates a comparable sensor response as the sensor response to a massive change of hydrogen concentration (e.g. 250 to 500 ppm).

In US 2014/0 379 299 A1 the volume change is optically detected. An optic grating is made from palladium containing material. At hydrogen concentrations in the security relevant range (0,4; 2%-4% respectively) the sensor response is small and the response time is equally on the order of minutes.

US 2004/0 173 004 A1 discloses a modification of a palladium based sensor by means of a thin cover layer made of zeolite which covers the sensor has a pore size of 2.85 angstrom to 3 angstrom and is about 20 microns thick. Optical as well as resistive measurement of the palladium element below the zeolite layer are discussed. The zeolite layer should make the sensor selective with respect to hydrogen and less sensitive towards reducers (carbon monoxide, hydrocarbons etc.). The very small pores of the zeolite should only allow hydrogen to diffuse to the sensor. The application of such thick layers may cause a massive loss in quality of the quartz oscillator elements. In addition zeolite itself absorbs gases which may cause a superposition of signals. Such sensors are thus also less attractive.

There is a need for a sensor material which partially or completely overcomes these drawbacks and alloys a more reliable, rapid and robust sensing of hydrogen in gases and liquids.

It is the object of the invention to improve sensor elements for sensing hydrogen.

The object is achieved by the subject-matter of the independent claims. Preferred embodiments are subject-matter of the dependent claims.

The invention provides a sensor layer system precursor configured for forming a sensor layer system, the sensor layer system being configured for absorbing hydrogen, the sensor layer system precursor including a sensing layer precursor made from a sensing layer precursor material that consists of:
20% by weight to 90% by weight palladium or palladium alloy the palladium alloy consisting of palladium and at least one palladium alloy partner chosen from group VIIIB, wherein the amount-of-substance fraction of palladium is at least 85% and the sum of the amount-of-substance fractions of all palladium alloy partners contained in the palladium alloy is at most 15% with respect to the whole amount of substance of the palladium alloy, respectively;
10% by weight to 80% by weight sacrificial metal, the sacrificial metal being at least as electropositive as palladium and each palladium alloy partner and/or the sacrificial metal being selectively transformable by a chemical process into a soluble and/or ionic form;
remainder unavoidable impurities; and
optionally up to and including 30% by weight pore filler precursor metal that is transformable into a pore filler by means of a pore filler reaction component.

Preferably, the palladium alloy is a single phase palladium alloy.

Preferably, each palladium alloy partner is chosen from a group comprising gold, iridium, copper, nickel, platinum, rhodium, ruthenium and silver.

Preferably, the pore filler precursor metal is chosen from a group comprising zinc and copper.

Preferably, the sacrificial metal of the sensing layer precursor material is chosen from a group comprising aluminum, cobalt, iron, lithium, zinc and alkaline earth metals, preferably calcium or magnesium or mixtures thereof, as well as copper, nickel and silver, wherein copper, nickel and silver are only chosen, if they are not chosen as a palladium alloy partner.

Preferably, the sensor layer system precursor further comprises a cover metal layer precursor that is applied to at least one side of the sensing layer precursor and that is made from a cover metal layer precursor material that consists of:
at least 40% by weight silver, gold, or silver-gold-alloy consisting of silver, gold and unavoidable impurities;
10% by weight to 60% by weight sacrificial metal, the sacrificial metal being at least as electropositive as each other constituent of the cover metal layer precursor material and/or the sacrificial metal being selectively transformable by a chemical process into a soluble and/or ionic form;
remainder unavoidable impurities; and
optionally up to and including 50% by weight palladium;
optionally up to and including 27% pore filler precursor metal, that is transformable into a pore filler by means of a pore filler reaction component.

Preferably, the sensor layer system precursor further comprises a metal base layer precursor that is applied to the lower side of the sensing layer precursor and that is made from a metal base layer precursor material that consists of:
at least 40% by weight silver, gold, or silver-gold-alloy consisting of silver, gold and unavoidable impurities;
10% by weight to 60% by weight sacrificial metal, the sacrificial metal being at least as electropositive as each other constituent of the metal base layer precursor material and/or the sacrificial metal being selectively transformable by a chemical process into a soluble and/or ionic form;
remainder unavoidable impurities; and
optionally up to and including 50% by weight palladium;
optionally up to and including 27% pore filler precursor metal, that is transformable into a pore filler by means of a pore filler reaction component.

Preferably, the sensor layer system precursor further comprises a terminal metal layer precursor that is applied to the upper side of the sensing layer precursor and that is made from a terminal metal layer precursor material that consists of:
at least 40% by weight silver, gold, or silver-gold-alloy consisting of silver, gold and unavoidable impurities;
10% by weight to 60% by weight sacrificial metal, the sacrificial metal being at least as electropositive as each other constituent of the terminal metal layer precursor material and/or the sacrificial metal being selectively transformable by a chemical process into a soluble and/or ionic form;
remainder unavoidable impurities; and
optionally up to and including 50% by weight palladium;
optionally up to and including 27% pore filler precursor metal, that is transformable into a pore filler by means of a pore filler reaction component.

Preferably, the sensor layer system precursor comprises a bonding agent layer that is the lowermost layer and consists of a bonding agent, wherein preferably the bonding agent is tantalum.

Preferably, the sensor layer system precursor comprises a connecting layer that directly connects the bonding agent layer with the cover metal layer and/or the sensing layer in a direct manner.

Preferably, the sensor layer system precursor comprises a second terminal layer that is the uppermost layer.

The invention provides a sensor layer system for a hydrogen sensor element configured for sensing a hydrogen concentration of, preferably non-bound, hydrogen in a fluid, the sensor layer system being configured for absorbing hydrogen, the sensor layer system being manufacturable from a preferred sensor layer system precursor by selectively removing sacrificial metal, preferably from the sensing layer precursor, in such a way that the sensor layer system includes a porous sensing layer generated from the sensing layer precursor.

Preferably, the sensing layer has pores that at least partially include a pore filler material that is chosen from a group comprising zinc, copper, nano porous material, MOF, copper oxide, copper doped calcium phosphate hydroxyapatite, cerium oxide, praseodymium oxide, iron, gold nano-particles, transitional metals, transitional metal oxides, rare earth metal oxides, manganese, cerium oxide, praseodymium oxide ore copper doped apatite and phosphates, silicates, carbonates, preferably of transitional metals or rare earth metals.

Preferably, the sensing layer has a layer thickness from 50 nm to 500 nm, preferably from 50 nm to 400 nm, preferably from 200 nm to 400 nm, preferably from 200 nm to 300 nm.

Preferably, the porosity of the sensing layer and/or the cover metal layer and/or the metal base layer and/or the terminal metal layer is more than 30% by volume and less than 100% by volume of the respective layer.

Preferably, the average pore diameter of the sensing layer and/or the cover metal layer and/or the metal base layer and/or the terminal metal layer is from 5 nm to 30 nm, preferably from 10 nm to 20 nm.

The invention provides a hydrogen sensor element for a hydrogen sensor device for sensing a concentration of, preferably non-bound, hydrogen in a fluid, the hydrogen sensor element comprising at least one oscillating member and a preferred sensor layer system arranged on a portion of the oscillating member.

The invention provides a manufacturing method for manufacturing a sensor layer system for a hydrogen sensor element that is configured for a hydrogen sensing device for sensing a concentration of, preferably non-bound, hydrogen in a fluid, the method comprising:
providing a preferred sensor layer system precursor; and
selectively removing sacrificial metal from the sensor layer system precursor in order to form pores.

The invention provides a manufacturing method for manufacturing a hydrogen sensor element for a hydrogen sensor device for sensing a concentration of, preferably non-bound, hydrogen in a fluid, the method comprising:
providing an oscillating member;
applying a preferred sensor layer system precursor to the oscillating member; and
selectively removing sacrificial metal from the sensor layer system precursor in order to form pores, preferably such that a preferred sensor layer system is obtained.

The invention provides a use of a preferred sensor layer system on an oscillating member or a bending oscillating member so as to detect a hydrogen concentration of a fluid.

A basic idea of the invention relates to a porous metal layer for detecting hydrogen in a gas or liquid. Also porous metal layers that have an increased silver or gold concentration may be provided on one or both sides on the surface of the porous metal layer. A method for manufacturing such porous metal layers, hydrogen sensors having such a porous metal layer and sensing methods for determining the hydrogen concentration in a gas or liquid using such sensors is discussed.

Further ideas of the invention relate to a device and a method for determining critical hydrogen concentrations in gases, in particular in the field of explosive limits of hydrogen air mixtures, in the field of hydrogen storage, generation and use in the field of energy and transport and in monitoring technical resources.

Preferred aspects of the invention relate to
(i) a device for determining hydrogen concentration in a gas comprising an oscillating member that is laden with a porous metal layer,
(ii) an electronic component, preferably for evaluating the oscillating behavior of the oscillating member,
(iii) a housing having at least one opening, and
(iv) a membrane for covering the opening of the housing.

Preferred aspects of the invention relate to a method for determining the safety of gases, in particular air/gas mixtures, wherein the at least one above described porous metal layer on the oscillating member is brought in to contact with the gas or liquid to be measured via the opening in the housing, and the oscillating behavior is sensed by electronics.

Preferred further aspects of the invention relate to a method for monitoring technical resources such as lubricants, motor oils, heat transfer liquids, wherein the above membrane separates the monitored liquid from the interior of the sensor housing. A gas remains in the interior of the housing that is in exchange via the membrane with the liquid.

Other preferred aspects relate to sensors with porous metal layers, wherein the pores of the metal layer are partially or completely filled with suitable materials. Thereby specific sensors may be obtained having improved stability or faster response behavior at increasing or decreasing hydrogen concentration.

A porous well-defined palladium layer having high mechanical stability and optional filler in the metal pores made of suitable filler material is formed for absorbing hydrogen. The layers can be used as reactive components of hydrogen sensors by means of suitable sensing methods for mass, thickness, mechanical tension or pressure, or volume change.

A porous layer is generally described by
(i) the thickness, which can be measured by means of suitable optical (microscopy, specifically electron microscopy) methods along a section or break perpendicular to the layer;
(ii) the diameters of the pores, which can be measured by means of suitable and known per se methods. Specifically, electron microscopy is a suitable optical method. Typically the average pore diameter is meant, which is calculated via the average value after measuring a suitable number of pores by singular measurement.
(iii) the fraction of volume of pores of the entire layer. This so called porosity describes the ratio of empty fractions of volume to fractions of volume filled with solid material. Typical porosity is a few percent (e.g. voids or errors in a solid), some ten percent (typical porosity of a layer formed from particles, loose fillings of particles) or very large (above 80%) with insulating materials or foams. The volume fraction of the pores can be determined via the density of the porous layer or from the number and size of the pores in a representative number of measurements along breaking edges or cross-sections of the material.

A designated herein, a layer is called well-defined, if with representative handling of the layer for relevant periods of time the layer does not suffer significant decomposition and has clearly defined geometric extent. Representative handling may include vibrations, impacts, temperature changes and other forms of stress, which can affect a part during its use. This is particularly relevant for parts that are employed in industry or on machines, such as for monitoring motor cooling and lubrication, heat transfer liquids, electrical plants or in vehicles. Suitable descriptions of the mechanical stress factors are known by the skilled person and depend on the field of application. A relevant time period depends on the time period of application of the part. Decomposition processes can manifest in mechanical changes such as cracks, loss of parts of the layer material, delamination of the layer material from the substrate structure and other forms of decomposition.

A clearly defined geometric extent of a porous layer as described herein is given, if the lower and/or upper side of the porous layer are mostly flat, such that they may basically described as a plane. The roughness of the well-defined porous layer is thus smaller as the average pore diameter of the pores of the layer. Suitable measurement methods for determining surface roughness of a layer are known by the skilled person.

Manufacture of the inventive layers can be achieved in multiple steps:

### Step 1

In step 1 preferably thick (typically more than 100 nm thick compared to otherwise used palladium containing layers), preferably massive (neglectable porosity below 5%) palladium containing so-called starting metal layers are applied to a suitable substrate, such as oscillating members, quartz crystals or the like. Before that one or more intermediate layers may be applied that can improve bonding to the substrate. A suitable intermediate layer for palladium is, for example, a 2 nm tantalum layer. Chromium or titanium containing layers are also possible.

The starting metal layer later serving as sensing layer includes:
(a) at least 20 percent by weight palladium,
(b) optionally an alloy partner for palladium from a group of the elements copper, platinum, silver, gold, ruthenium, rhodium, iridium, nickel, as well as
(c) at least one sacrificial metal and
(d) optionally a pore filler precursor metal.

The starting metal layers can be used for manufacture of porous palladium containing layers and are a precursor to that.

The sacrificial metal is included in the metal layer with about 10 to 80 percent by weight. The sacrificial metal should meet at least one of the following conditions. The sacrificial metal is
(i) more electropositive than palladium and each alloy partner (standard reduction potential with respect to hydrogen electrode of the sacrificial metal is lower than the standard reduction potential with respect to hydrogen electrode of palladium and each alloy partner); and/or
(ii) the sacrificial metal is distinguished from palladium and each alloy partner such that the sacrificial metal may be selectively transformed into a soluble and/or ionic form by an additional chemical process, wherein it is removed from the metal layer. If copper, silver or nickel are not used as an alloy partner, they may also be used as sacrificial metals having specific chemical processes.

In addition to the sacrificial metal optionally a pore filler precursor metal may be used, wherein the pore filler precursor metal forms 1 to 30 percent by weight of the metal layer. The pore filler precursor metal can be transformed into a pore filler by means of at least one reaction component.

Particular useful pore filler precursor metals are zinc and copper, for example. Reaction components may be multiply substituted carboxylic acids or nitrogen bases, such as imidazoles.

Specifically suitable pore fillers are nano porous materials, in particular MOFs (metal organic framework materials) due to them being configurable by means of a defined pore size to limit the access of gases into the interior of the palladium containing layer. Specifically suitable MOF are zinc or copper containing MOF, such as ZIF-8. Suitable reaction components for forming MOF compounds are sufficiently known.

Other suitable pore fillers are compounds that can react with hydrogen, in particular oxidizing oxides and phosphates of transition metals, particularly rare earth metals. Without being a binding theory, it is supposed that the pore filler can consume hydrogen in the interior of the layer, for example, by means of catalytic oxidation and thus the response time of the sensor is improved for decreasing hydrogen concentrations. Examples for suitable pore filler are copper oxide, copper doped calcium phosphate hydroxyapatite or cerium oxide.

Methods for applying such metal layers are known per se and include among others methods for deposition in liquid or gas phase.

Methods for applying metal layers in liquids include electro plating, chemical metal deposition (also called electroless plating) and combinations of the methods as well as the use of multistep methods, wherein the metals can be applied together or in sequence. Optionally, in between or at the end—according to the desired result—heating of the layer may facilitate mixing of the metals or demixing of the metals.

Metal deposition in the gas phase is often preferred and possible by sputtering, physical vapor deposition (PVD), decomposition of suitable chemical compounds (also known as metal organic chemical vapor deposition or MOCVD) and other gas or vacuum deposition methods which are used in metal surface processing and treatment. Furthermore, thin film deposition methods may be used that are sufficiently known in manufacturing technology for semiconductor elements and electronic parts.

In usual application, the manufacture of the metal layer in the gas phase is advantageous since the type and combination of the different metals (palladium, alloy partners, sacrificial metal, optional pore filler precursor material) is usually simpler.

In certain situations it may be advantageous to use a hybrid of methods or deposit a part of the components as particles on the part, respectively, and to link them through further metal deposition with further metal.

Preferably, subsequent to depositing the starting metal layer or sensing layer, respectively, an additional terminal metal layer or cover metal layer is applied. This layer is described in more detail below and may improve quality and robustness of the metal layer or the layer system, respectively. Use of a pore filler may additionally allow the terminal layer to improve long term stability of the sensors in applications with high concentration of organic compounds, particularly if the operating temperature is high.

In certain situations it may be advantageous to provide a base layer or cover layer made from a porous noble metal before applying the porous metal layers. This layer may be applied to the intermediate layer or directly on the oscillating member. Preferably, suitable metal deposition methods are used, wherein at the start of the metal deposition a mixture of a sacrificial metal and a noble metal is used, that is unreactive with hydrogen but may be alloyed with palladium. Suitable noble metals are for example gold and/or silver. Subsequent to applying a preferably 10 nm to 50 nm thick metal base layer, the deposition method may be gradually adapted to the composition of the porous sensing layer. Alternatively, depending on the composition one may change directly to the composition of the sensing layer. Use of a gradient may be advantageous since tensions in the material may be avoided. The resulting sensor and resulting layer system respectively may thus be more stable.

As before and similar to the sensing layer and starting layer, respectively, in order to manufacture the metal base layer, the sacrificial metal is removed from the base metal layer and forms a porous noble metal base layer.

The porous metal layer may be separated from the oscillating member by the metal base layer or cover metal layer respectively and/or from the environment by the terminal metal layer or cover metal layer respectively. Both surface neighboring parts of the metal layer have at least one of the following features:
(i) there is hardly no or no palladium included and the palladium content is less than 50% by weight;
(ii) at least one sacrificial metal and optionally a pore filler precursor metal are included, wherein the content of sacrificial metal is at least ⅓ of the content of sacrificial metal in the starting metal layer or sensing layer respectively; and/or
(iii) there are at least 40% by weight silver or gold or alloys thereof.

Preferably thin metal base and terminal layers (cover metal layers) are provided whose thickness is preferably less than a quarter of the total thickness of the layer system, preferably less than a tenth of the total thickness of the layer system.

Preferably the applied metal layers are consolidated by heating. The temperature and duration of this heat treatment particularly depends on the metals used, their fraction and the metal deposition method. When transitional metals are used as sacrificial metal, the use of heat treatment can be forgone. In using main group elements a heat treatment is usually advantageous.

When oscillating members such as quartz oscillating members are used it may be advantageous to provide a metal base layer and a terminal metal layer in exploiting the mass effect due to hydrogen absorption. In applying the porous palladium (alloy) layers to another sensor substrate, such as a bending or tensile sensor, it may be advantageous to only use a terminal metal layer or cover layer respectively.

When calcium or magnesium is used as sacrificial metal and particularly in chemical vapor deposition of the metal layers, the duration and temperature of the subsequent treatment can be used to set the pore size.

### Step 2

In step 2 the layer system may be subjected to a chemical process that partially or completely removes the sacrificial metal from the metal layers. Without being held to the theory, it is assumed that the remaining metals that stay in the layers are reorganized and a new metal mixture may be formed. Therein a suitable pre-processing of the layer and/or suitable pairs of sacrificial metals and palladium or palladium/alloy partner or palladium/alloy partner/pore filler precursor metal may lead to a porous metal layer, wherein the kind and nature of the pores may be partially controlled by the conditions of the chemical process and also the choice of metal.

Suitable chemical processes for selectively removing a sacrificial metal are known per se and are also called dealloying. An example for a suitable sacrificial metal is cobalt that may be removed in an acidic regime with dilute sulfuric acid in an electrochemical cell. Other sacrificial metals are, for example, zinc or iron, aluminum, lithium and alkaline earth metals or mixtures thereof. Earth alkaline metals are advantageous since they may be removed in pH neutral regimes, in particular by chelating agent such as ethylenediaminetetraacetic acid (EDTA) and other methods. Preferred earth alkaline metals are calcium and magnesium.

The chemical processes usually take place in aqueous baths in which the layer system is immersed. In general suitable processes for a particular choice of sacrificial metal/palladium alloy are advantageous, when the processes may selectively exploit the chemical distinctions of the sacrificial metal and the palladium or palladium alloy. The variety of the processes is thus large. Subsequently some advantageous combinations are listed:

For cobalt and nickel an electrochemical treatment in acidic baths may be advantageous. Optionally aids such as surface active substances or salts may be added to the baths.

For zinc acidic baths with or without additional electrochemical treatment may be used. Typical baths include at least 0.1 mols sulfuric acid per liter; heating the bath may be advantageous.

For aluminum strongly alkaline baths (more than 1 mol/l NaOH or KOH) and increased temperatures (more than 90° C.) may be used, so as to remove the sacrificial metal from the metal layer. Since many oscillating members are only partly alkaline proof, aluminum is less suitable as a sacrificial metal yet still usable. A preferred use of aluminum is in manufacturing sensors for bending sensors made from stainless steel. Here a strongly alkaline bath can be used due to the sensor material being well enduring.

For lithium removal is possible under mild conditions (few or no addition of acid for treatment bath required) and moderate temperature (below 60° C.).

For calcium and magnesium removing is under mild conditions is also possible. Use of buffered solutions of chelating agents such as EDTA is advantageous and allows in particular removal of calcium or magnesium from the metal layers.

If very reactive metals are used, is can be advantageous to expose the metal layers to humid air with optionally increased temperature before application of the bath. This can facilitate the later formation of good pores.

Optionally the layer(s) formed in this manner can be consolidated by thermal or chemical methods. An additional electrochemical or current less deposition of small amounts of palladium or other components of the system may be used since this can consolidate the layer structure system or the initially formed porous metal structure respectively. The additionally deposited metal in this step is preferably less than 20% of the total metal of the layer system, preferably less than 10%. Thermal post-processing are known per se and can be used to consolidate newly formed structures.

Suitable baths for post treatment of the layer structure system with additional palladium include for example 0.01 to 1 g/l Pd(NH₃)₄Cl₂, 5 to 40 g/l Na₂EDTA, 20 to 200 ml ammonia solution (29 mol-% NH₃ in water) per liter and 0.01 to 1 ml hydrazine solution (1 mol/l in water) and are preferably applied with slightly increased temperatures.

### Step 3 (Optional)

In step 3, in case a pore filler precursor metal was used, the reaction component can be brought into contact with the porous layer(s) of step 2 at suitable reaction conditions (temperature, solvent, reaction time). Thereby the pore filler is preferably formed in the interior of the pores of the metal layers. Preferably at least 90% by weight of the totally formed pore filler is formed within the pores of the metal film and is stuck therein. In some embodiments it can be advantageous to introduce additional components into the above reaction mixture.

Suitable reaction components for manufacture of pore filler based on MOF include the necessary ligands, typically carboxylic acids or organic bases or complex forming ligands in concentrations of 0.05 bis 5 g/l in buffered aqueous solutions with a changing part of short-chain alcohols, like methanol, glycol or ethanol or in pure organic solvents. The reaction component can be slightly heated in this solution. The reaction components used are distinguished from reaction components for manufacturing MOF materials particularly in that there are no or almost no metal cation necessary for forming MOF (distinction 1) and/or that they are of low concentration (distinction 2).

In preferred embodiments the sacrificial metal and the pore filler precursor metal can be the same. This is advantageous because less materials are needed. This embodiment may also be described as having a partial removal of sacrificial metal in step 2 and, particularly ensuing, reaction of the left-over sacrificial metal with a reaction component of step 3. This embodiment, in case of zinc (sacrificial metal and pore filler precursor metal are the same) or copper (alloy partner, sacrificial metal and pore filler precursor metal are the same), of preferred interest since zinc and copper are also applicable in the manufacture of suitable MOF. In case of copper this is a very elegant choice since very few elements need to be used.

In specific embodiments step 2 and 3 may be combined, and the removal of the sacrificial metal and forming of the pore filler can be performed in the same step.

In an embodiment porous metal layers are applied to an oscillating member for detecting hydrogen. To this end quartz oscillating members or bending sensors are coated with a porous metal layer by using step 1 with or without pore filler precursor metal. Preferably the coating is applied by sputtering, thermally post-processed and transformed into a porous metal layer by means of a mild reaction solution.

Specifically preferred metal layers can be applied along the total cross-section. Preferably all metal layers save for the interlayer are porous and comprise
a silver rich or gold rich metal base layer or cover metal layer with a layer thickness of 10 nm to 100 nm, preferably 10 nm to 80 nm, more preferably 10 nm to 50 nm;
a palladium rich layer with at least 70% by weight palladium and a preferred layer thickness of 50 nm to 500 nm, preferably of 50 nm to 400 nm, preferably of 200 nm to 400 nm, preferably of 200 nm to 300 nm;
a silver rich or gold rich terminal metal layer or cover layer with a layer thickness of 10 nm to 100 nm, preferably 10 nm to 80 nm, more preferably 10 nm to 50 nm.

The metal layers are preferably applied to a quartz oscillating member.

Deposition of the metal layer(s) can be performed by sputtering. Corresponding devices are obtainable by AJA International Inc., North Scituate, Mass., or Aurion Anlagentechnik GmbH.

Suitable quartz oscillating members are obtainable from Micro Crystal Switzerland, Lap-Tech Inc or Geyer Electronics. Preferably the quartz oscillating member is directly coated as part of a specialize manufacture, particularly before and after applying electrodes to the quartz oscillating member.

Preferably the following metals are deposited, particularly in this sequence:
1) interlayer: depositing of a 1 nm to 5 nm, preferably 2 nm interlayer, preferably tantalum, on the quartz;
2) depositing of 2 nm to 10 nm, preferably 5 nm, pure silver or gold, particularly as connection between interlayer and porous metal layer and/or corrosion protection. This is also called second interlayer;
3) depositing of 10 nm to 30 nm, preferably 20 nm, of a silver/sacrificial metal or gold/sacrificial metal composition, e.g. 70% to 85% silver and/or gold as well as 15% to 30% sacrificial metal;
4) depositing of 50 nm to 300 nm, preferably of 50 nm to 250 nm, preferably of 100 nm to 250 nm, preferably of 100 nm to 200 nm, palladium/silver/sacrificial metal composition (70% to 85% of palladium alloy from 85% palladium/15%) silver; 15% to 30% sacrificial metal)
5) depositing of 10 nm to 30 nm, preferably 20 nm, of a gold/sacrificial metal composition (70-85% Au; 30-15% sacrificial metal)

Preferably the metal layers are heated after sputtering, preferably under inert gas, and brought to step 2 preferably after cooling.

A preferred sacrificial metal is calcium or magnesium.

The coated quartz oscillating members are transformed into a porous form by means of treatment in a chemical process. A preferred chemical process includes contacting the metal layers with a solution having the following components:
(a) 20 g/l disodium ethylenediaminetetraacetate (Na₂EDTA);
(b) 0.1 g to 5 g of non-ionic surfactant, preferably based on ethylene glycol or a wetting agent or other surface active substance or more than 20 g of monoalkyloligoethylenglycol, such as diethylenglycolmonobutylether.

A slightly increased temperature of the bath of 50° C. is preferred. Subsequently the layers are cleaned, preferably multiple times, with highly purified water and/or isopropyl alcohol, and dried and tempered, particularly under nitrogen, at preferably 100° C. to 250° C. With this the manufacture of the porous metal layers is complete.

After cooling the parts can be completed, calibrated and used as a sensor for hydrogen.

In an embodiment porous metal layers having pore filler precursor metal are applied to the quartz oscillating members and transformed into porous palladium layers or palladium alloy layers having pore filler precursor metal by means of steps 1 and 2. The pore filler precursor metal can remain in form of layers or particle in the interior of the pores on the inner surface thereof or be partially or completely dissolved in the palladium or palladium alloy. In the former case, step 3 contacting with the reaction component leads to a transformation of the pore filler precursor metal within the pores into the pore filler material. In the latter case the presence of the reaction component leads to a migration of the pore filler precursor metal from the palladium or palladium alloy matrix into the pores and subsequent reaction with the reaction component under formation of the pore filler material that precipitates within the pores.

It is preferred that the formation of the pore filler only takes place, if the reaction component can diffuse to the corresponding location in the pore. Thus the process of pore filling can be carried out in a widely self-controlled manner due to the pores filling with pore filler such that, when the remaining cavities or cracks between pore filler material and palladium or palladium alloy matrix becomes narrow, no further reaction component fits therebetween. Since crystallization of MOFs from less structured or amorphous precursor products typically coincides with volume expansion the forming MOF crystals can funnel into the metal matrix and form densely packed pores. This is advantageous because the use of MOF as selective pore materials may be improved. Larger hydrocarbons or functionalized organic compounds thus can no longer penetrate into the interior of the sensing layer. Without being held to the theory, it is assumed that this effect leads to an improved selective detection of hydrogen in such systems.

A preferred material can be manufactured thusly:

Step 1: the metals are applied by sputtering, particularly in this sequence, to an oscillating member:

1) interlayer: depositing of 1 nm to 5 nm, preferably 2 nm of interlayer, preferably tantalum, on the quartz;
2) depositing of 2 nm to 10 nm, preferably 5 nm, of pure gold
3) depositing of 10 nm to 30 nm, preferably 20 nm, of a gold/sacrificial metal mixture (70% to 90% gold; 10% to 30% sacrificial metal)
4) depositing of 50 nm to 300 nm, preferably of 50 nm to 250 nm, preferably of 100 nm to 250 nm, preferably of 100 nm to 200 nm palladium/silver/sacrificial metal/pore filler precursor metal mixture (60% to 80% of a palladium alloy of 85%) palladium/15% silver; 10% to 30% sacrificial metal; 1% to 15% zinc)
5) depositing of 10 nm to 30 nm, preferably 20 nm, of a gold/sacrificial metal mixture (70% to 90% gold; 10% to 30% sacrificial metal)

The metal layers are heated after sputtering, preferably under an inert gas and are brought to step 2 after cooling.

Step 2: the coated quartz oscillating members are transformed into their porous form by treatment with a chemical process. A preferred chemical process includes contacting the metal layers with a solution including the following composition:

(a) 50 g/l ethanoic acid;
(b) 0.1 g to 5 g of a non-ionic surfactant, preferably based on ethylene glycol or a wetting agent or other surface active substance or more than 20 g of a short chained monoalkyloligoethylenglycol such as diethylenglycolmonobutylether.

After that the layers are rinsed, preferably multiple times, with particularly highly purified water and/or isopropyl alcohol and dried and tempered, preferably under nitrogen, at 250° C.

Step 3: The coated oscillating member is preferably contacted with a war solution of the following composition:

(a) 10 g/l to 80 g/l 2-methylimidazole methanol
(b) 5 g to 50 g sodium formate per liter methanol
and subsequently intensely rinsed, particularly multiple times, with pure methanol. The parts are tempered preferably under nitrogen and at preferably 120° C. and are completed after cooling and can be used as sensors. Optionally 5 nm to 25 nm gold can be deposited on the material, e.g. by means of a vacuum deposition method. This means a partial or complete manufacture of the terminal layer after completing step 3. This variant usually does not cause complete closure of the pores since the gold is not able to close off the pores with such a low occupancy so that still a good gas transport takes place.

In one embodiment porous metal layers with a specific pore filler precursor metal are applied to the oscillating members and transformed by steps 1 and 2 into porous palladium or palladium alloy layers with pore filler precursor metall. The pore filler precursor metal as metal or metallic derivative can remain in the interior of the pores on the pore inner surface in the form of layers or particles or can exist partially or completely dissolved in the palladium or palladium alloy. (Air) oxygen, an oxidizer such as hydrogen peroxide or further reactants such as phosphate containing buffers and/or other additives are used as a reaction component. The contacting of reaction components causes a transformation of the pore filler precursor metal and forms the pore filler material that precipitates in the interior of the pores. Suitable pore filler materials can be materials that are able to oxidize hydrogen, such as copper oxide, copper doped calcium hydroxyapatite or rare earth metal oxides such as cerium oxide or praseodymium oxide.

Sensors with such pore filler are specifically suitable for detecting initial occurrence of traces of hydrogen in an otherwise hydrogen free or oxygen free environment. Without being held to the theory, it is assumed that the availability of an oxidizer as a solid within the pores (above pore filler materials) allows reaction of hydrogen bound to palladium or palladium alloy with the oxidizer and thus locally forms water or other reaction products, wherein preferably the palladium or palladium alloy surface of the porous metal matrix is freed so as to absorb additional hydrogen from the environment. This leads to a higher and more rapid hydrogen absorption in the layers with pore filler compared to layers without pore filler. These layers are particularly suitable for one-time use for monitoring high value process gases.

Sensors with pore filler can be used in oxygen containing environment such that the mass increase due to hydrogen is amplified, for example, by additional absorption of oxygen since the pore filler material can be oxidized by absorbing oxygen after its reduction. In such sensor materials it is preferred that the pore filler material does not entirely fill the pores so as to allow sufficient gas transport into the whole layer. Specifically suited layers are particularly iron or copper oxide containing pore filler materials, optionally having gold nanoparticles. Without being held to the theory, it is assumed that the local vicinity of palladium surface with adsorbed hydrogen and reactive oxide surface (iron, copper and/or other transition metals) of the pore filler material cause a rapid reaction of hydrogen to water. Since oxygen (atomic weight 16 g/mol) is much heavier than hydrogen (atomic weight 1 g/mol), this oxidation of the hydrogen bounded or adsorbed to the palladium can cause a mass increase of up to 8 times (water H₂O is formed from two hydrogen atoms and one oxygen atom), and thereby allows an improved sensor sensitivity.

Sensors of this kind can be manufactured as follows:

Step 1: The metals are applied to the oscillating members by sputtering, particularly in this sequence:

1) interlayer: depositing of 1 nm to 5 nm, preferably 2 nm interlayer, preferably tantalum, on the quartz;
2) depositing of 5 nm to 20 nm, preferably 10 nm, or pure gold;
3) depositing of 10 nm to 30 nm, preferably 20 nm, of a gold/sacrificial metal mixture (60% to 90% gold; 10% to 40% sacrificial metal)
4) depositing of 50 nm to 300 nm, particularly of 50 nm to 250 nm, particularly of 100 nm to 250 nm, particularly of 100 nm to 200 nm palladium/gold/sacrificial metal/pore filler precursor metal mixture (60% of a palladium alloy of 85%) palladium/15% gold; 15% sacrificial metal; 25% cerium)
5) depositing of 10 nm to 30 nm, preferably 20 nm, of gold/sacrificial metal mixture (60% to 90% gold; 10% to 40% sacrificial metal)

The metal layers are heated preferably after sputtering and particularly under inert gas and brought to step 2 after cooling.

Step 2: the coated quartz oscillating members are transformed into their porous form by treatment with a chemical process. A preferred chemical process includes contacting the metal layers with a solution including the following composition:

(a) 50 g/l ethanoic acid;
(b) 0.1 g to 5 g of a non-ionic surfactant, preferably based on ethylene glycol or a wetting agent or other surface active substance or more than 20 g of a short chained monoalkyloligoethylenglycol such as diethylenglycolmonobutylether.

After that the layers are rinsed, preferably multiple times, with particularly highly purified water and/or isopropyl alcohol and dried and tempered, preferably under nitrogen, at 250° C.

Step 3: The coated oscillating member is preferably contacted with a solution of the following composition:

(a) 10 g hydrogen peroxide solution (35% in water) per liter water
and subsequently preferably multiple times particularly with pure water intensely rinsed. The parts are tempered preferably under nitrogen at particularly 250° C. and completed after cooling and can be used as sensors.

In an embodiment the sensors can be accommodated in a housing, wherein particularly additionally at least one heating element is provided. Suitable heating elements are known per se and can be operated by current or irradiation with light, preferably infra-red light. In a preferred embodiment the porous palladium containing metal layer can be deposited in such a manner that the metal layer itself can be heated by applying electrical current, whereby absorbed and adsorbed gases can be removed from the layer. Periodic supply of current to the layer can improve the sensitivity. Suitable embodiments are known per se, wherein the geometry of coated oscillating members is also known.

In an embodiment a part or the whole electrode that is usually deposited on the quartz oscillating member, can be replaced by the porous palladium containing layer. Such sensor can be manufactured in few steps, wherein the excitation of oscillation as well as read out and mass change of the oscillating member (actual effect as sensor for hydrogen) can take place via the same material. This elegant execution is possible due to the robust construction and reliable function of the composition of the metal layer(s) used herein.

A variety of commercially available quartz oscillating members are procurable in a variety of electrode configurations. The manufacturing process of the quartz oscillators is changed in the area of electrode deposition such that
(i) under use of the same or similar geometries (dimension and kind of the used masks, path and form of electrodes)
(ii) under use of similar metal deposition methods (usually in vacuum)
   metal compositions of the kind described herein are deposited instead of the platinum or palladium alloys used in manufacturing of traditional quartz oscillators.

At least one additional process step (designated <<step 2>> above) is preferably inserted into the manufacturing process so as to transform the electrodes into porous metal.

Preferably only a part of the electrode material is immersed in the corresponding reaction solutions/baths of step 2.

It is particularly preferred to use the above process in a form in which soldering pads for contacting the oscillating members are configured such that porous parts of the electrode are arranged in majority on the oscillating part of the part.

In one embodiment a porous palladium containing layer can be deposited on a suitable oscillating member, wherein it is accommodated in a housing with an opening and a membrane. These sensors are particularly suitable for detecting hydrogen in demanding environments, particularly in the presence of increased temperature and/or the presence of other compounds, such as hydrocarbons, organic compounds, such as solvents, oils and/or foodstuff vapors, machine oils, fuels as well as electrolytes of fuel cell systems and batteries respectively. Polymer membranes can be used as membrane, through which hydrogen is able to diffuse through. Suitable polymers are known as high performance polymers with high chemical and mechanical stability. Suitable polymers are among others polyimides, polyethers and polyetherketones as well as fluorous polymers such as poly (vinylidenfluorid). In monitoring hydrogen concentration in organic liquids (above oils, electrolytes, heat transfer oils etc.) choosing the membrane in respect to stability is relevant. Suitable materials are sufficiently known.

Embodiments of the invention are described in more detail with reference to the accompanying schematic drawings. Therein:

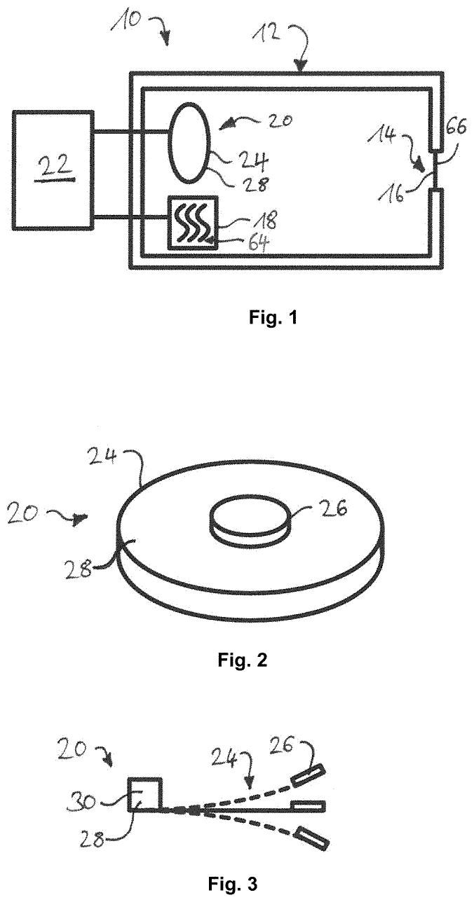

FIG. 1 depicts an embodiment of a hydrogen sensor device;

FIG. 2 depicts an embodiment of a hydrogen sensor element;

FIG. 3 a further embodiment of a hydrogen sensor element;

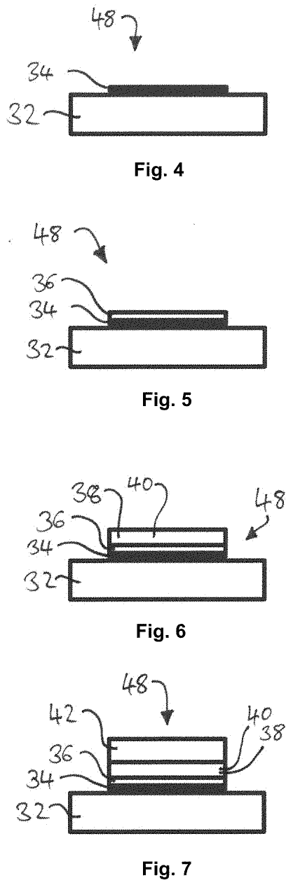

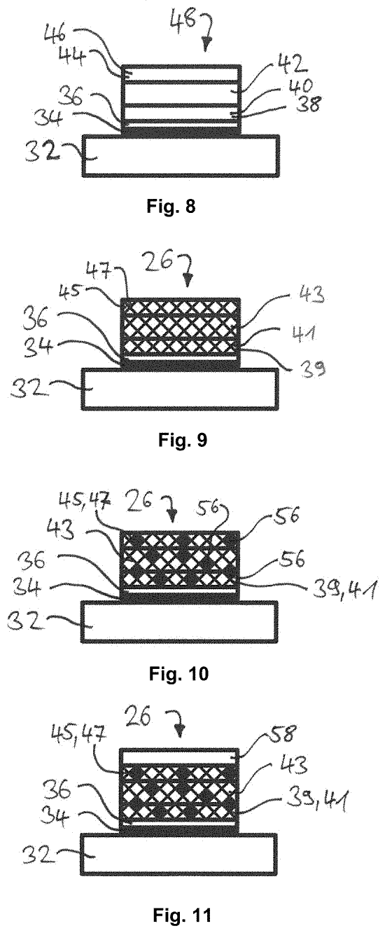

FIG. 4 to FIG. 9 an embodiment of a manufacturing method for a hydrogen sensor element;

FIG. 10 an embodiment of a hydrogen sensor element;

FIG. 11 an embodiment of a hydrogen sensor element;

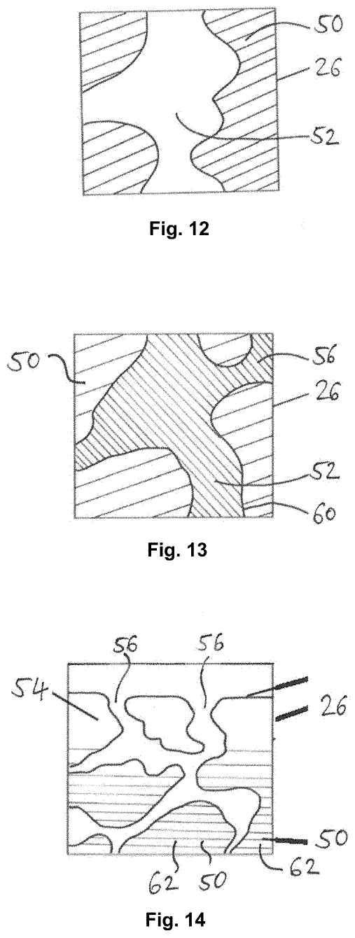

FIG. 12 a section of the sensor layer system of FIG. 9;

FIG. 13 a section of the sensor layer system of FIG. 10; and

FIG. 14 a section of the sensor layer system of FIG. 11.

Initial reference is made to FIG. 1 showing an embodiment of a hydrogen sensor device 10 that is configured for sensing hydrogen concentration. The hydrogen sensor device 10 comprises a housing device 12. The housing device 12 is provided with a housing opening 14. The housing opening 14 can be closed by a membrane 16 that is configured such that hydrogen may diffuse through the membrane 16. Furthermore, a heating device 18 can be arranged within the housing device 12.

In addition, a hydrogen sensor element 20 is arranged within the housing device 12. The hydrogen sensor element 20 can be heated by the heating device 18.

The hydrogen sensor device 10 further comprises a control device 22 that can control the sensing process. The control device 22 can also be partially accommodated within the housing device 12. The control device 22 is configured for controlling the heating device 18 and the hydrogen sensor element 20, particularly for activating the heating device 18 in order to heat the hydrogen sensor element 20 and/or for determining the hydrogen concentration sensed by the hydrogen sensor element 20.

As depicted in FIG. 2, the hydrogen sensor element 20 can comprise an oscillating member 24 on which a sensor layer device 26 is arranged. The sensor layer device 26 will be discussed in more detail later. The oscillating member 24 is, for example, a quartz oscillating member 28 an can be excited to oscillate by means of electrical (alternating) current. The oscillation frequency of the oscillating member 24 depends on the geometry (here: cylindrical) of the oscillating member 24 and the configuration of the sensor layer system 28. Other geometries are also usable, such as a tuning fork geometry.

The sensor layer system 28 is configured such that it can particularly absorb unbound hydrogen. Thereby the mass of the sensor layer system 26 is increased and the oscillating frequency of the oscillating member 24 changes depending on the mass change. This change of the oscillating frequency can be detected by the control device 22 and be translated into a value corresponding to the hydrogen concentration.

As depicted in FIG. 3, the hydrogen sensor element 20 may also have a bar like oscillating member 24. This oscillating member 24 can be made from stainless steel. Additionally, an actuator 30 can be provided that causes the oscillating member 24 to oscillate. The functional principle, however, is the same as in the embodiment of FIG. 2.

Referring now to FIG. 4 through FIG. 9, manufacture of the sensor layer system 26 is depicted schematically. The layers are preferably sputtered; although other depositing processes may be used.

Initially a bonding agent layer 34 made of a bonding agent is preferably deposited on a substrate layer 32 that particularly may be formed by the oscillating member 24. The bonding agent layer 34 has a layer thickness between 1 nm and 5 nm. Tantalum is preferably used as the bonding agent.

A connecting layer 36 made of connecting material having a layer thickness between 5 nm and 20 nm may be deposited on the bonding agent layer 34. Gold, silver or an alloy thereof may be preferably used as the connecting material.

A lower cover metal layer precursor 38 that is made from a cover metal layer precursor material having a layer thickness of 10 nm to 30 nm can be deposited on the connecting layer 36. The cover metal precursor material consists of at least 40 wt % silver, gold or silver gold alloy, wherein the silver gold alloy in turn consists of silver, gold and unavoidable impurities as well as 10 wt-% to 60 wt-% sacrificial metal that is at least as electropositive as each other component of the cover metal layer precursor material and/or wherein the sacrificial metal is selectively transformable by a chemical process into a soluble and/or ionic form, e.g. magnesium or calcium. The cover metal layer precursor material may also include up to and including 50 wt-% palladium.

The lower cover metal layer precursor 38 is also called metal base layer precursor 40. The cover metal layer precursor material is then called metal base layer precursor material.

Further, a sensing layer precursor 42 made from a sensing layer precursor material that has a layer thickness of 50 nm to 300 nm, preferably of 50 nm to 250 nm, preferably of 100 nm to 250 nm, preferably of 100 nm to 200 nm can be deposited on the lower cover metal layer precursor 38. The sensing layer precursor material consists of 20 wt-% to 90 wt-% palladium or palladium alloy, wherein the palladium alloy consists of palladium and at least one palladium alloy partner that is chosen from group VIIIB, wherein the mole fraction of palladium is at least 85% and the sum of the mole fractions of all palladium alloy partners included in the palladium alloy is at most 15% with respect to the total amount of substance of the palladium alloy as well as 10 wt-% to 80 wt-% sacrificial metal, wherein the sacrificial metal is at least as electropositive as palladium and each palladium alloy partner and/or wherein the sacrificial metal is selectively transformable by a chemical process into a soluble and/or ionic form.

An upper cover metal layer precursor 44 that is made from the cover metal precursor material with a layer thickness of 10 nm to 30 nm. The composition, however, may be different from the lower cover metal layer precursor 38. The upper cover metal layer precursor 44 is also called a terminal metal layer precursor 46. The cover metal layer precursor material is then called terminal metal layer precursor material.

The previously described metal layers (bonding agent layer 34, connecting layer 36, lower cover metal layer precursor 38, metal base layer precursor 40, sensing layer precursor 42, upper cover metal layer precursor 44, terminal metal layer precursor 46) need not all be present. Depending on the application single layers except for the sensing layer precursor 42 may be omitted. The metal layers collectively form a sensor layer system precursor 48 that can be transformed into the sensor layer system 26 as subsequently described.

In a chemical process, the sensor layer system precursor 48 is contacted with a solution that includes 20 g/l disodium ethylenediaminetetraacetate (Na₂EDTA) and 0.1 g to 5 g of a non-ionic surfactant, preferably based on ethylene glycol or a wetting agent or other surface active substance or more than 20 g of a monoalkyloligoethyleneglycol, such as diethyleneglycolmonobutylether. Preferably a solution bath formed from this solution is heated to a slightly increased temperature between 40° C. and 60° C., preferably 50° C.

With this the sacrificial metal is removed from the sensor layer system precursor 48, wherein the remaining components of the sensor layer system precursor 48 are deposited in a porous configuration. Subsequently this intermediate product may be rinsed multiple times with highly purified water and isopropyl alcohol and dried. Finally the result may be tempered under nitrogen at about 100° C. to 250° C.

Now the sensor layer system precursor 48 has become the sensor layer system 26. Therein the lower cover metal layer precursor 38 and the metal base layer precursor 40 were respectively transformed into a lower cover metal layer 39 and a metal base layer 41. The sensing layer precursor 42 has become the sensing layer 43 in which hydrogen can be well absorbed. The upper cover metal layer precursor 44 and the terminal metal layer precursor 46 have respectively become upper cover metal layer 45 and terminal metal layer 47.

The lower cover metal layer 39, the metal base layer 41, the sensing layer 43, the upper cover metal layer 45 and the terminal metal layer 47 are respectively porous due to removal of the sacrificial metal.

After cooling the sensor layer system 26 can be fixed to the oscillating member 24 so as to form the hydrogen sensor element 20. The hydrogen sensor element 20 can be calibrated and subsequently used as a sensor for hydrogen.

As depicted in a section in FIG. 12, the sensor layer system 26 has a porous configuration. The material is depicted in a cross-section, wherein there is a palladium rich portion 50 and a pore 52.

Subsequently further embodiments are explained insofar as they differ from the previous embodiments.

In addition to the metal layers explained so far, in this embodiment a pore filler precursor metal is deposited or sputtered. The pore filler precursor metal can be deposited onto the inner surface of the pores or at least partially be dissolved within the palladium containing layer.

In order to transform this sensor layer system precursor 48 into the sensor layer system 26, the already porous sensor layer system precursor is additionally brought into contact with a reaction component that is able to transform the pore filler precursor metal into a pore filler material or cause a migration of the pore filler precursor metal from the palladium containing layers into the pores 52 so as to form the pore filler material.

For the transformation into a porous form a solution of 50 g/l ethanoic acid and 0.1 g to 5 g of a non-ionic surfactant is used, preferably based on ethylene glycol or a wetting agent or other surface active substance or more than 20 g of a short chained monoalkyloligoethyleneglycol such as diethyleneglycolmonobutylether. After that the layers are rinsed with highly purified water and isopropyl alcohol multiple times and dried and tempered under nitrogen at 250° C.

For transforming or generating the pore filler material from the pore filler precursor metal the porous sensor layer system precursor 48 is brought into contact with a warm solution that includes 10 g to 80 g 2-methylimidazole per liter methanol and 5 g to 50 g sodium formate per liter methanol. Thus a sensor layer system 26 with pore filler material 56 is formed.

Subsequently, it is intensely rinsed with pure methanol, tempered under nitrogen at 120° C., and the hydrogen sensor element 20 is formed an used as a sensor after cooling.

Optionally, as depicted in FIG. 11, 5 nm to 25 nm gold may be deposited by means of a vacuum depositing method onto the sensor layer system 26. With this a second terminal layer 58 is formed that is not porous but massive. Yet there is no significant pore closure due to the gold not being able to close off the pores entirely due to the low occupancy. Consequently a good gas transport can still take place.

As depicted in a section of FIG. 13 in more detail, the sensor layer system 26 comprises a porous configuration with pore filler material 56. The material is depicted in a cross-section wherein palladium rich portions 50 and the pore 52 are present. The pore 52 is filled with pore filler material 56 that is in contact with the palladium rich portion 50 via contact surface 60.

In a variant oxygen from the air, an oxidizer such as hydrogen peroxide or phosphorous buffers or other additives are used as reaction components in generating the pore filler material 56 from the pore filler precursor metal. The contact with the reaction components leads to a transformation of the pore filler precursor metal and formation of the pore filler material 56 that precipitates in the interior of the pores 52. Suitable pore filler materials can be materials that are able to oxidize hydrogen, such as copper oxide or copper doped calcium hydroxyapatite, or rare earth metal oxides such as cerium oxide praseodymium oxide.

In this variant a 10 g hydrogen peroxide solution (35% in water) per liter water is used as a reaction component.

As depicted in a section of FIG. 14 in more detail, the sensor layer system 26 can have a porous configuration in an embodiment. This again is a section of a cross-section. The palladium rich portion 50, a palladium poor or free portion 54 and the pore 52 are present, wherein the palladium rich portion 50 is a central portion 62 of the sensor layer system 26. Consequently in this embodiment the palladium concentration of the metal layers may continuously increase from the surface (FIG. 14, at the top) towards the bottom till the sensing layer 43.

Referring now to FIG. 1, the heating device 18 has at least one heating element 64. Suitable heating elements are sufficiently known and can produce heat via current or irradiation with light, specifically infrared light. The sensor layer system 26 is preferably attached to the oscillating member 24 such that it can be heated by applying a current, and thus absorbed and adsorbed gases may be removed from the layer. Applying current to the layer periodically can increase the sensitivity of the hydrogen sensor element 20.

In a variant of the hydrogen sensor element 20 an electrode or a part of the electrode required for operating the oscillating member 24 can be replaced by the sensor layer system 26. The hydrogen sensor element 20 may thus be manufactured in fewer steps and oscillation excitation as well as read out of the mass change of the oscillating member 24 can take place through the same material due to the sensing effect for hydrogen.

The hydrogen sensor device 10 is suitable to detect hydrogen in demanding environments, particularly in the presence of increased temperatures and other compounds, such as hydrocarbons, organic compounds, specific solvents, oils, foodstuff vapors, machine oils, fuels and electrolytes of fuel cells and batteries.

The membrane 16 can be a polymer membrane 66 through which hydrogen may diffuse. Suitable polymers are known as high performance polymers having high chemical and mechanical stability. Suitable polymers are among others polyimides, polyethers, polyetherketones and fluorous polymers such as polyvinylidenfluoride. In monitoring the hydrogen concentration in organic liquids the choice of membrane 16 with respect to stability is relevant. Suitable materials are sufficiently known.

With the measures described herein, porous metal layers are provided that are palladium rich in the center portion and are covered at least on one side with a thin hydrogen resistant and porous noble metal layer. The porous metal layers have a clearly defined geometry and can thus be employed in demanding environments, for example, on vibrating systems for detecting hydrogen. Furthermore, methods for manufacturing the abovementioned systems and composites including the abovementioned porous metals and pore fillers are provided.

### LIST OF REFERENCE NUMERALS

10 hydrogen sensor device
12 housing device
14 housing opening
16 membrane
18 heating device
20 hydrogen sensor element
22 control device
24 oscillating member
26 sensor layer device
28 quartz oscillating member
30 actuator
32 substrate layer
34 bonding agent layer
36 connecting layer
38 lower cover metal layer precursor (metal layer)
39 lower cover metal layer
40 metal base layer precursor (metal layer)
41 metal base layer
42 sensing layer precursor (metal layer)
43 sensing layer
44 upper cover metal layer precursor (metal layer)
45 upper cover metal layer
46 terminal metal layer precursor (metal layer)
47 terminal metal layer
48 sensor layer system precursor
50 palladium rich portion
52 pore
54 palladium poor/palladium free portion
56 pore filler material
58 second terminal layer
60 contact surface
62 central portion
64 heating member
66 polymer membrane

1. A sensor layer system precursor (48) configured for forming a sensor layer system (26), the sensor layer system (26) being configured for absorbing hydrogen, the sensor layer system precursor (48) including a sensing layer precursor (42) made from a sensing layer precursor material that consists of:
20% by weight to 90% by weight palladium or palladium alloy, preferably a single phase palladium alloy, the palladium alloy consisting of palladium and at least one palladium alloy partner chosen from group VIIIB, wherein the amount-of-substance fraction of palladium is at least 85% and the sum of the amount-of-substance fractions of all palladium alloy partners contained in the palladium alloy is at most 15% with respect to the whole amount of substance of the palladium alloy, respectively;
10% by weight to 80% by weight sacrificial metal, the sacrificial metal being at least as electropositive as palladium and each palladium alloy partner and/or the sacrificial metal being selectively transformable by a chemical process into a soluble and/or ionic form;
remainder unavoidable impurities; and
optionally up to and including 30% by weight pore filler precursor metal that is transformable into a pore filler by means of a pore filler reaction component.

2. The sensor layer system precursor (48) according to claim 1, wherein each palladium alloy partner is chosen from a group comprising gold, iridium, copper, nickel, platinum, rhodium, ruthenium and silver.

3. The sensor layer system precursor (48) according to claim 1 or 2, wherein the sacrificial metal of the sensing layer precursor material (42) is chosen from a group comprising aluminum, cobalt, iron, lithium, zinc and alkaline earth metals, preferably calcium or magnesium or mixtures thereof, as well as copper, nickel and silver, wherein copper, nickel and silver are only chosen, if they are not chosen as a palladium alloy partner.

4. The sensor layer system precursor (48) according to any of the claims 1 to 3, wherein the pore filler precursor metal is chosen from a group comprising zinc and copper.

5. The sensor layer system precursor (48) according to any of the claims 1 to 4, further comprising a cover metal layer precursor (38, 44) that is applied to at least one side of the sensing layer precursor (10) and that is made from a cover metal layer precursor material that consists of:
at least 40% by weight silver, gold, or silver-gold-alloy consisting of silver, gold and unavoidable impurities;
10% by weight to 60% by weight sacrificial metal, the sacrificial metal being at least as electropositive as each other constituent of the cover metal layer precursor material and/or the sacrificial metal being selectively transformable by a chemical process into a soluble and/or ionic form;
remainder unavoidable impurities; and
optionally up to and including 50% by weight palladium;
optionally up to and including 27% pore filler precursor metal, that is transformable into a pore filler by means of a pore filler reaction component.

6. A sensor layer system (26) for a hydrogen sensor element (20) configured for sensing a hydrogen concentration of, preferably non-bound, hydrogen in a fluid, the sensor layer system (26) being configured for absorbing hydrogen, the sensor layer system (26) being manufacturable from a sensor layer system precursor (48) according to any of the preceding claims by selectively removing sacrificial metal, preferably from the sensing layer precursor, in such a way that the sensor layer system (26) includes a porous sensing layer (43) generated from the sensing layer precursor (42).

7. The sensor layer system (26) according to claim 6, further comprising a cover metal layer (39, 45) that is manufacturable by selectively removing sacrificial metal, preferably from the cover metal layer precursor (38, 44), such that the cover metal layer (39, 45) is generated on at least one side of the sensing layer (43).

8. The sensor layer system (26) according to claim 6 or 7, wherein the sensing layer (43) has pores (52) that at least partially include a pore filler material (56) that is chosen from a group comprising zinc, copper, nano porous material, MOF, copper oxide, copper doped calcium phosphate hydroxyapatite, cerium oxide, praseodymium oxide, iron, gold nano-particles, transitional metals, transitional metal oxides, rare earth metal oxides, manganese, cerium oxide, praseodymium oxide ore copper doped apatite and phosphates, silicates, carbonates, preferably of transitional metals or rare earth metals.

9. The sensor layer system (26) according to any of the claims 6 to 8, wherein the sensing layer (43) has a layer thickness from 50 nm to 500 nm, preferably from 50 nm to 400 nm, preferably from 200 nm to 400 nm, preferably from 200 nm to 300 nm.

10. The sensor layer system (26) according to any of the claims 6 to 9, wherein the porosity of the sensing layer (43) and/or the cover metal layer (39, 45) and/or the metal base layer (41) and/or the terminal metal layer (47) is more than 30% by volume and less than 100% by volume of the respective layer.

11. The sensor layer system (26) according to any of the claims 6 to 10, wherein the average pore diameter of the sensing layer (43) and/or the cover metal layer (39, 45) and/or the metal base layer (41) and/or the terminal metal layer (47) is from 5 nm to 30 nm, preferably from 10 nm to 20 nm.

12. A hydrogen sensor element (20) for a hydrogen sensor device (10) for sensing a concentration of, preferably non-bound, hydrogen in a fluid, the hydrogen sensor element (20) comprising at least one oscillating member (24) and a sensor layer system (26) according to any of the claims 6 to 11 arranged on a portion of the oscillating member (24).

13. A manufacturing method for manufacturing a sensor layer system (26) for a hydrogen sensor element (20) that is configured for a hydrogen sensing device (10) for sensing a concentration of, preferably non-bound, hydrogen in a fluid, the method comprising:
providing a sensor layer system precursor (48) according to any of the claims 1 to 5; and
selectively removing sacrificial metal from the sensor layer system precursor (48) in order to form pores (52).

14. A manufacturing method for manufacturing a hydrogen sensor element (20) for a hydrogen sensor device (10) for sensing a concentration of, preferably non-bound, hydrogen in a fluid, the method comprising:
providing an oscillating member (24);
applying a sensor layer system precursor (48) according to any of the claims 1 to 5 to the oscillating member (26); and
selectively removing sacrificial metal from the sensor layer system precursor (48) in order to form pores (52), preferably such that a sensor layer system (26) according to any of the claims 6 to 11 is obtained.

15. A method of using a sensor layer system (26) according to any of the claims 6 to 11 on an oscillating member (24) or a bending oscillating member so as to detect a hydrogen concentration of a fluid.
